(19) United States
(12) Patent Application Publication
(10) Pub. No.: US 2025/0258087 A1
(43) Pub. Date: Aug. 14, 2025

(54) POTASSIUM MEASUREMENT WITH SODIUM GLUCONATE

(71) Applicant: Hach Company, Loveland, CO (US)

(72) Inventor: Devin Lee Maurer, Ames, IA (US)

(21) Appl. No.: 18/440,378

(22) Filed: Feb. 13, 2024

(51) Int. Cl. G01N 21/31; G01N 33/18

(52) U.S. Cl. CPC G01N 21/31; G01N 33/18

(57) ABSTRACT

An embodiment provides a method for measuring potassium in a sample, including: chelating an interferant, using an amount of sodium gluconate, in the sample; mitigating an ammonium interference, using an amount of alkali hydroxide, in the sample; and measuring, using a tetraphenylborate indicator, the amount of potassium in the sample by measuring a change in intensity of the absorbance of the chelated sample. Other aspects are described and claimed.

### BACKGROUND

This application relates generally to measuring potassium in a sample, and, more particularly, to measuring potassium in the presence of a sodium gluconate chelator.

Ensuring water quality is critical in a number of industries such as pharmaceuticals and other manufacturing fields. Additionally, ensuring water quality is critical to the health and well-being of humans, animals, and plants which are reliant on the water for survival. Analytes such as potassium may be measured and monitored. Potassium level outside of acceptable parameters in water can be harmful to humans or animals. For example, potassium may cause the water to be less desirable to consumers or facilities. Potassium may be present from natural or human activities such as manufacturing. Measurement and mitigation of potassium may result in higher costs of water treatment. Therefore, detecting the presence and concentration of potassium in water or other liquid solutions is vital.

### BRIEF SUMMARY

In summary, one embodiment provides a method for measuring potassium in a sample, comprising: chelating an interferant, using an amount of sodium gluconate, in the sample; mitigating an ammonium interference, using an amount of alkali hydroxide, in the sample; and measuring, using a tetraphenylborate indicator, the amount of potassium in the sample by measuring a change in intensity of the absorbance of the chelated sample.

Another embodiment provides a measurement device for measuring potassium in a sample, comprising: a prepackaged module, wherein the prepackaged module comprises an amount of sodium gluconate; a processor; and a memory storing instructions executable by the processor to: chelate an interferant, using the amount of sodium gluconate, in the sample; mitigate an ammonium interference, using an amount of alkali hydroxide, in the sample; and measure, using a tetraphenylborate indicator, the amount of potassium in the sample, using a photometric device, by measuring a change in intensity of the absorbance of the chelated sample.

A further embodiment provides a product for measuring potassium in a sample, comprising: a prepackaged module, wherein the prepackaged module comprises an amount of sodium gluconate; a storage device that stores code, the code being executable by a processor and comprising: code that chelates an interferant, using the amount of sodium gluconate, in the sample; code that mitigates an ammonium interference, using an amount of alkali hydroxide, in the sample; and code that measures, using a tetraphenylborate indicator, the amount of potassium in the sample, using a photometric device, by measuring a change in intensity of the absorbance of the chelated sample.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1A illustrates and chemical reaction of ammonium to ammonia.

FIG. 1B illustrates an example chelation of calcium with sodium gluconate.

FIG. 1C illustrates an example chelation of magnesium with sodium gluconate.

FIG. 2 illustrates a flow diagram of an example potassium measurement method.

FIG. 3 illustrates example data of mitigation of magnesium and ammonium with sodium gluconate.

FIG. 4 illustrates example data of the accuracy of sodium gluconate mitigation.

FIG. 5 illustrates example data of the accuracy and precision of a sodium gluconate mitigation with ammonium, calcium, and magnesium interferences.

FIG. 6 illustrates example real world data from different samples comparing the sodium gluconate method to a previous method.

FIG. 7 illustrates an example of computer circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

Potassium measurement in water or other aqueous samples or solutions is important for many different reasons. For example, potassium measurement may be used to determine the quality of water. High concentrations of potassium may be harmful to animals, humans, and/or plants. Accordingly, as another example, a user or entity may want the potassium in a body of water to be under a particular threshold, therefore, the user may measure the potassium in order to determine if the amount of potassium is under that threshold. Potassium may be present in a body of water either naturally or from human activity such as manufacturing or storage conditions. Also, a level of potassium may need to be monitored and/or controlled in a solution for human consumption, medical, industrial, food/beverage, or manufacturing applications.

Conventional methods of potassium measurement and detection may have limitations discussed herein. For example, conventional methods may use formaldehyde. Governments may have limits of use or waste disposal requirements for this conventional method. One example method is the Hach 8049 method. This method may detect potassium in the 0.1-700 mg/L range. The conventional method for potassium concentration determination uses formaldehyde to mitigate ammonium interference. For example, ammonium reacts with tetraphenylborate in the same manner as potassium resulting in turbidity, by reacting with ammonium to result in hexamethylenetramine that will not react with tetraphenylborate. EDTA (ethylenediaminetetraacetic acid) is currently used to mitigate magnesium interference (also reacts with tetraphenylborate resulting in turbidity) by forming a soluble complex with the magnesium that will not react with the tetraphenylborate. In an embodiment, the tetraphenylborate is an indicator for an amount of potassium in the sample. The method uses either a spectrophotomic or gravimetric measurement.

However, there are some limitations with this method. Mainly, the method uses formaldehyde. At the very least, formaldehyde may cause irritation of the skin, eyes, nose, and throat. Formaldehyde is also a known carcinogenic substance. Therefore, the reduction or complete elimination of the use of formaldehyde is beneficial to users of a product, and reduces cost, storage, and disposal of entities using the product. Current methods, systems, and kits for potassium measurement using the above method involve hazardous or controlled reagents make obtaining the reagents and disposal difficult. Since, potassium ions are commonly found in various water matrices, and selective mitigation of potassium in presence of other analytes can be difficult, a method to measure potassium in the presence of interferants is needed. What is needed is an accurate method to measure potassium in a sample with fewer interference concerns and less hazardous reagents.

Accordingly, an embodiment provides a system and method for measuring potassium in the presence of an interferant. In an embodiment the interferant may be ammonium, calcium, and/or magnesium. In an embodiment, hazardous reagents such as formaldehyde are not used. In an embodiment, a colorimetric method may be used to potassium in an aqueous sample or solution. The aqueous sample may contain an amount of potassium to be measured. The aqueous sample may be buffered. The buffering may be to a pH to shift an equilibrium between ammonium and ammonia to favor ammonia to reduce interference and accuracy of a potassium measurement. The aqueous sample may be chelated. In an embodiment, sodium gluconate may be used as a chelating agent chelating. In an embodiment, the amount of potassium in an aqueous sample may be measured by measuring a change in intensity of the absorbance of the chelated aqueous sample. In an embodiment, change in intensity of the absorbance is proportional to a concentration of the amount of potassium in the aqueous sample. In an embodiment, the potassium chelating agent may be introduced to the aqueous sample in a form selected from the group consisting of: a solution, a powder, and a prepackaged module. In an embodiment, the aqueous sample may comprise interferences, which may include: ammonium, calcium, magnesium, or the like. In an embodiment, the aqueous sample may be a sample of water for quality testing. The system and method may use colorimetric and/or spectrophotometric measurement. The method may use lithium hydroxide in place of the formaldehyde to mitigate the ammonium interference by increasing the pH thus pushing the ammonium to ammonia, which will not react with the tetraphenylborate. At the higher pH EDTA needed to be replaced in order to mitigate magnesium interference. Sodium gluconate was determined to be the best replacement for EDTA in mitigating magnesium at the higher pH.

The illustrated example embodiments will be best understood by reference to the figures. The following description is intended only by way of example, and simply illustrates certain example embodiments.

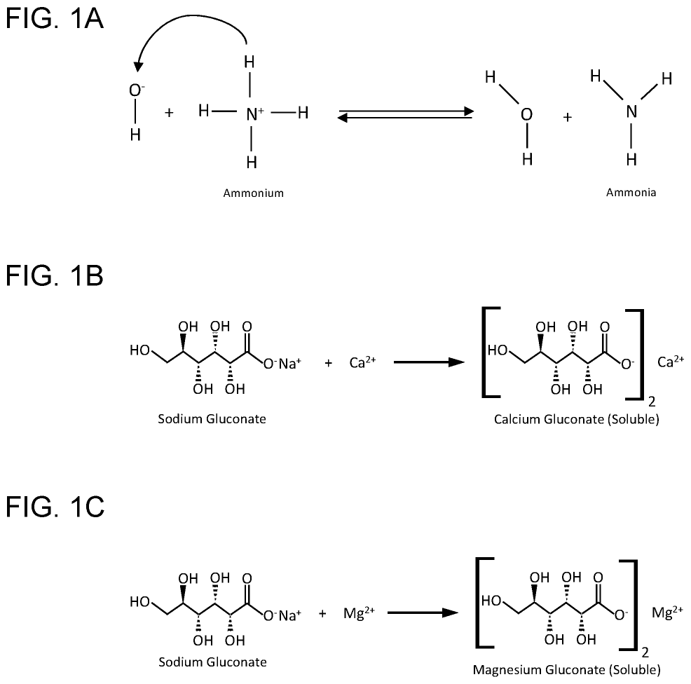

Referring to FIG. 1, in an embodiment, reaction schematics are illustrated. FIG. 1A illustrates a conversion of ammonium to ammonia. FIG. 1B illustrates a reaction of sodium gluconate with calcium in a sample to form calcium gluconate. FIG. 1C illustrates a reaction of sodium gluconate with magnesium in a sample to form magnesium gluconate. In an embodiment, sodium gluconate may be added to a sample containing potassium for a measurement of potassium as described herein.

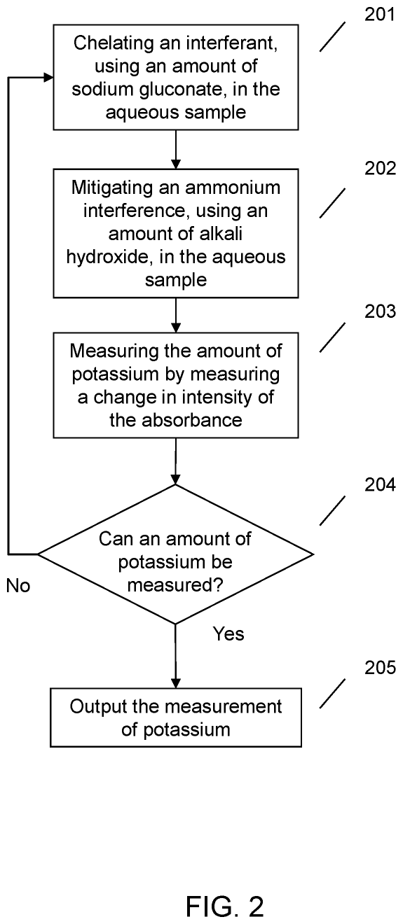

Referring to FIG. 2, an example system and method for measurement of potassium in an aqueous sample, sample, or solution is illustrated. In an embodiment, the aqueous sample may be pH adjusted. In an embodiment, a potassium chelating agent may be added to the aqueous sample. Different concentrations of potassium may result in different colorimetric intensities after respective chelation, the change in the colorimetric intensity may be correlated to a concentration of potassium in the aqueous sample.

Referring to FIG. 2. at 201, in an embodiment, a chelating agent may be added to the aqueous sample. The chelating agent may be added after a mitigation agent and/or a buffering agent. In an embodiment, the chelating agent may chelate an interferant. For example, an interferant may be magnesium, calcium, or the like. The chelating agent may be sodium gluconate. The sodium gluconate in the presence of calcium or magnesium may form either calcium gluconate (See FIG. 1B) or magnesium gluconate (See FIG. 1C).

The amount of sodium gluconate added to the sample may be adjusted in concentration based upon a concentration of an interferant present in the sample. In an embodiment, 0.25 g of sodium gluconate may be used to mitigate interferences at the proposed pH, of calcium (up to 7000 mg/L Ca as CaCO₃) and magnesium (up to 6000 mg/L Mg as CaCO₃) in a 25 mL sample at room temperature. The gluconate forms a soluble complex with calcium and/or magnesium so that they do not react with the tetraphenylborate or excess hydroxide ions, both of which would result in white precipitate that would interfere with the potassium measurement. The chelating agent may be added to a chamber, vessel, or measurement chamber. The chelating agent may be added in a measured amount using manual or autonomous methods to correspond to the volume of the aqueous sample containing potassium.

In an embodiment, a sample or aqueous sample may be prepared. Reagents for mitigation, chelation, indicator, or measurement may be placed in a solution, aqueous sample, water sample or the like. The sample may be added, with other components, to a chamber, vessel, or the like as a powder, a liquid, or a prepackaged module. The sample and/or components may be added manually or using an autonomous system. In other words, the reagents for the method may be prepackaged and/or premeasured for ease of use. The prepackaged reagents may be added to a sample, or the sample may be added to a prepackaged reagent container or vial. Therefore, tetraphenylborate, sodium gluconate, and/or lithium hydroxide may be in prepackaged modules or a plurality of prepackaged modules for combination with a sample with potassium to be measured.

In an embodiment, the aqueous sample may be buffered or adjusted to a pH value. In an embodiment, a pH value may be selected to minimize interferences. For example, a pH may be selected based upon the concentration and/or composition of interferences. Possible interferences may include ammonium, magnesium, or the like. An aqueous sample may have may contain one or more interferences of varying concentration, or no interferents at all.

The aqueous sample may include a sample from a natural body of water, a holding tank, a processing tank, a pipe, or the like. The solution may be in a continuous flow, a standing volume of liquid, or any combination thereof. In one embodiment, the solution may be introduced to a reducing agent or a buffer, for example, a test chamber of the measurement device. In an embodiment, the measurement device may be a benchtop, field, or hand-held device. A hand-held device may have advantages such as lower cost, portability, field use, or the like. Introduction of the solution into the measurement device may include placing or introducing the solution into a test chamber manually by a user or using a mechanical means, for example, gravity flow, a pump, pressure, fluid flow, or the like. For example, a water sample for potassium measurement may be introduced to a measurement or test chamber using a pump. In an embodiment, valves or the like may control the influx and efflux of the solution into or out of the one or more chambers, if present.

A chamber, vessel, cell, chamber, or the like may contain an aqueous sample and associated reagents such as a reducing agent, a buffering agent, and/or a potassium chelating agent. Various reagents may be added to an aqueous sample in the form of a powder, a liquid, a prepackaged module, or the like. A device may contain one or more bottles of reagents which contain necessary reagents. The reagents contained in the one or more bottles may be pump fed or gravity fed. The flow of the reagents may be metered to ensure proper volume delivery to the measurement cell. The aqueous sample may be fed through a pressured inlet, a vessel, or the like. The aqueous sample may be introduced into the measurement chamber by a pump or gravity fed. The sampling device may be in series or parallel to an aqueous flow. The device may have a system to ensure proper mixing of the aqueous sample with a reducing agent, a buffering agent, and/or potassium chelating agent.

Additionally or alternatively, the measurement device may be present or introduced in a volume of the solution. The measurement device may then be exposed to the volume of an aqueous sample where it may perform measurements. The system may be a flow-through system in which an aqueous sample and/or reagents are automatically mixed and measured. Once the sample is in contact with the measurement system, the system may measure the potassium or an amount of potassium of the sample, as discussed in further detail herein. In an embodiment, the measurement device may include one or more chambers in which the one or more method steps may be performed.

In an embodiment, a sample with potassium to be measured may be a predetermined volume or brought to a predetermined volume with deionized water. Reagents described herein may be added to this volume. There may be more than one reagent added at different points in the measurement process. In other words, a first reagent may be added, the sample mixed, then a second reagent added, and then mixed once again. Mixing may be accomplished by mechanical inversion, stirring, pumping, or the like. Mixing may be performed at intermediate steps in between the addition of different reagents.

Referring to FIG. 2 at 202, in an embodiment, an ammonium interference may be mitigated. The mitigation may be accomplished by the addition of an alkali hydroxide to the aqueous sample. In an embodiment, the alkali hydroxide may be lithium hydroxide (LiOH) or sodium hydroxide (NaOH). Referring to FIG. 1A, in an embodiment, a reaction schematic of ammonium to ammonia is illustrated. In an embodiment, approximately 0.05 grams of LiOH may be added to a sample to mitigate 15 mg/L of ammonium nitrogen in a 25 mL sample at room temperature, and to a pH of approximately above a pH of 12. The hydroxide ion (OH⁻) reacts with the positively charged ammonium (NH⁺) resulting in two neutral species. Since the Ammonia is not positively charged it will not react with the tetraphenylborate and interfere with a measurement like the ammonium ion would. The reaction occurs quickly. The sample may be shaken or stirred after the hydroxide addition, or after any of the steps. An addition of greater than 0.1 g of LiOH to a 25 mL sample results in the formation of MgOH, in the presence of magnesium, which is not soluble. The resulting white precipitate will then also interfere with the measurement if the traditional EDTA is used as a mitigator for its direct interference.

Referring to FIG. 2. at 203, in an embodiment, the system and method may measure an amount of potassium in the solution by measuring a change in the intensity of the absorbance caused by the potassium reacting with the chelating agent. In an embodiment, the presence of potassium in an aqueous solution may cause an increase in absorbance intensity. Thus, the change in absorbance of the solution may be proportional to the amount of potassium within the solution. The sodium gluconate may remove calcium or magnesium interference from a potassium measurement. Accordingly, a measurement device or user can correlate the measured change in absorbance with the amount of potassium in the aqueous sample. In an embodiment, colorimetric techniques may measure a concentration of the potassium.

For example, the measuring may be a measurement of an absorbance at a wavelength for a colored complex. The measuring may include taking a ratio of absorbance collected at multiple wavelengths. Different measurement devices may be used to perform the measurement, for example, a portable parallel analyzer (PPA, such as the SL1000 available from Hach Company, Loveland, CO), test strips, colorimetric analyzers, spectrophotometers, pocket colorimeters, online process instruments, and the like.

Therefore, the absorbance intensity, of an aqueous sample containing potassium may be correlated to the concentration of the potassium in the aqueous solution. In an embodiment, the amount of absorbance may be proportional to an amount or concentration of potassium in the solution. Absorbance curves may be generated for a range of potassium concentrations, for any different condition that may affect absorption or absorbance values (e.g., temperature, sample content, turbidity, viscosity, measurement apparatus, aqueous sample chamber, etc.), or the like. The absorbance curves can then be used for determining the amount of potassium in the solution.

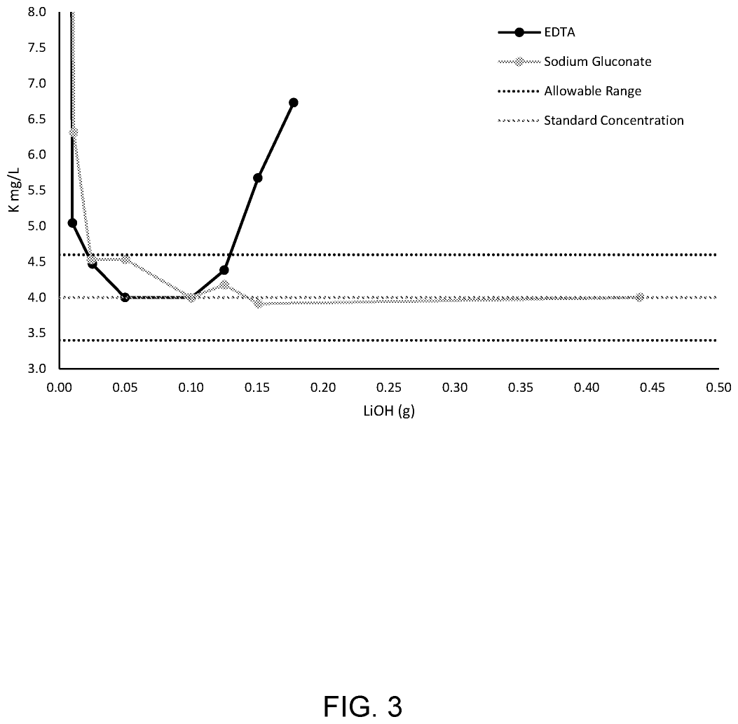

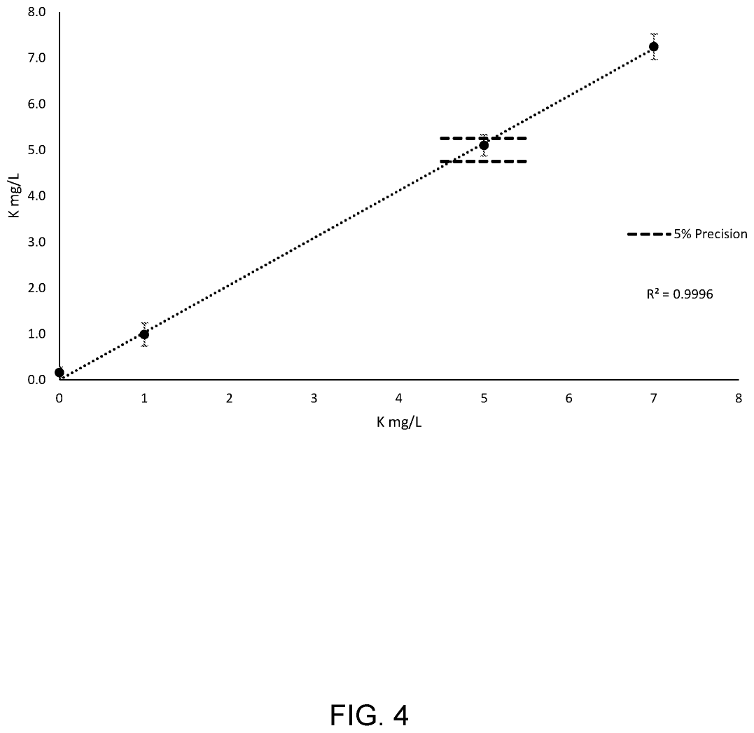

Referring to FIG. 3, in an embodiment, some example data of chelating in a potassium measurement method are illustrated. For example, note the EDTA data which goes out of the acceptable range (dotted lines). The EDTA departure from the acceptable range occurs around 0.15 g of lithium hydroxide (LiOH) and when potassium levels reach approximately 4.5 mg/L. In contrast, chelation with sodium gluconate maintains measurement of potassium levels, even in the presence of LiOH for the range illustrated. Therefore, sodium gluconate allows for a wider range and better accuracy of potassium measurement in the presence of an interferant such as magnesium. Referring to FIG. 4, in an embodiment, sample data is illustrated to demonstrate the precision of the method.

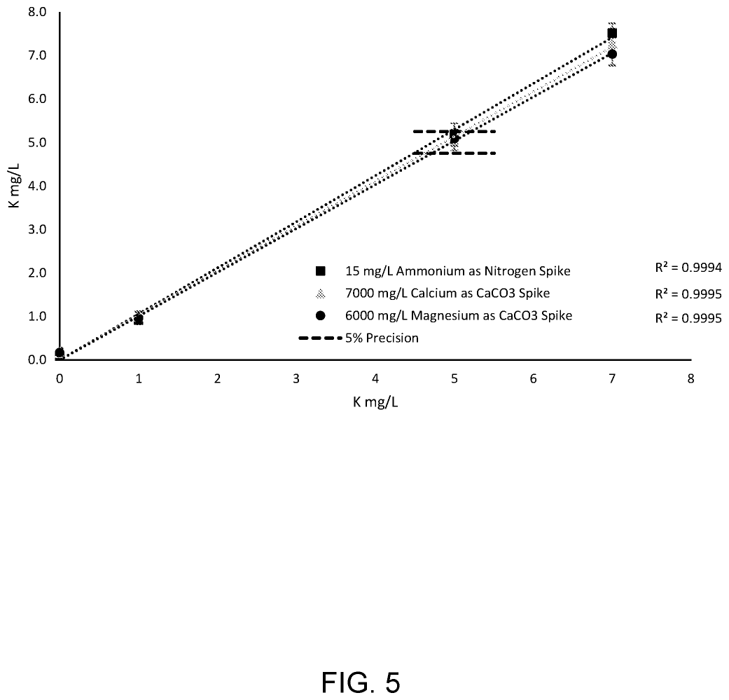

Referring to FIG. 5, in an embodiment, example data are illustrated using sodium gluconate as a chelator in the measurement of potassium. Three experimental conditions are shown. First, a 15 mg/L ammonium nitrogen spike. Second, a 7,000 mg/L calcium as calcium carbonate (CaCO₃) spike. Third, a 6,000 mg/L magnesium as CaCO₃ spike. These three conditions represent the three interferences that may prevent an accurate potassium measurement using other methods. Note that all three conditions and the three interferences do not prevent accurate measurement of potassium across the ranges illustrated.

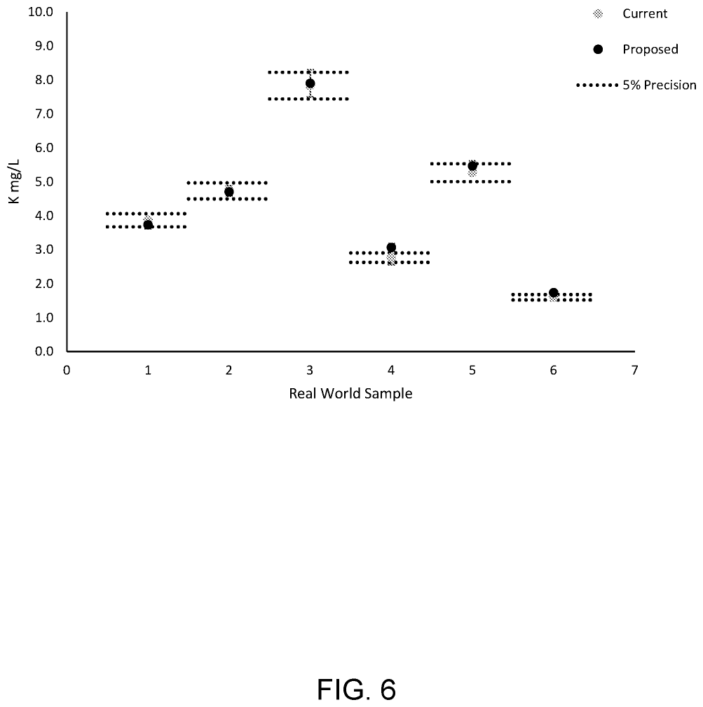

Referring to FIG. 6, in an embodiment, real world samples from various sites are collected. These real world samples are used as measurement samples for potassium comparing the conventional method described above, and the sodium gluconate method. The sodium gluconate method was within 0.6 to 11% without the use of formaldehyde for various real world examples.

At 204, in an embodiment, the system and method may determine if an amount of potassium may be measured. For example, an amount of potassium may be measured using spectrophometric methods using a spectrophotometer. For example, a turbidity of the sample may be measured after the disclosed method is performed. As an example, the Hach TU5 Series or Hach TL23 Series Laboratory Turbidimeters may be used available from Hach Company, Loveland CO, USA (TU5 Series is a registered trademark of Hach Company in the United States and other countries). Alternatively, a gravimetric method may be used by filtering out tetraphenylborate/potassium complex and weighing the complex. The absorbance measurements may be compared to expected values, historical values, or the like. Potassium measurement using spectrophometric methods may be at periodic intervals set by the user or preprogrammed frequencies in the device. Measurement of potassium by a device allows for real time data with very little human involvement in the measurement process. In the event that the system outputs an unexpected value, the system may automatically request re-measurement of a solution or sample.

A programmed calibration curve may be entered into the device for calibrating the measurement device. In an embodiment, the system and method may be periodically tested using a known amount of potassium in the sample. The system may then recalibrate or send an error report for maintenance. In the event that the error is caused by an unclean device or that the device otherwise needs cleaned, the system may implement a cleaning cycle. Cleaning of the spectrophotometer or measurement chamber may be required at an unspecified time interval, after a certain number of measurements, upon user or system request, or the like. In an embodiment, a cleaning cycle of the measurement device may be performed using either automated or manual methods.

At 204, in an embodiment, if a concentration of potassium cannot be determined, the system may continue to measure potassium and/or an absorbance signal. Additionally or alternatively, the system may output an alarm, log an event, or the like. If a concentration of potassium can be determined, the system may provide a measurement of potassium concentration at 205. The measurement which may be the absorbance intensity or potassium concentration may be an output that is provided to a device in the form of a display, printing, storage, audio, haptic feedback, or the like. Alternatively or additionally, the output may be sent to another device through wired, wireless, fiber optic, Bluetooth®, near field communication, or the like.

An embodiment may use an alarm to warn of a measurement or concentration outside acceptable levels. An embodiment may use a system to shut down water output or shunt water from sources with unacceptable levels of potassium. For example, a potassium measuring device may use a relay coupled to an electrically actuated valve, or the like. The system may connect to a communication network. The system may alert a user or a network. This alert may occur whether a potassium measurement is determined or not. An alert may be in a form of audio, visual, data, storing the data to a memory device, sending the output through a connected or wireless system, printing the output or the like. The system may log information such as the measurement location, a corrective action, geographical location, time, date, number of measurement cycles, or the like. The alert or log may be automated, meaning the system may automatically output whether a correction was required or not. The system may also have associated alarms, limits, or predetermined thresholds. For example, if a potassium concentration reaches a threshold. Alarms or logs may be analyzed in real-time, stored for later use, or any combination thereof.

Absorbance may be measured using different methods. For example, the measurement may be made in a vial or cuvette. As another example, the absorbance measurement may be performed using a micro or mesofluidic device, such as a “lab on a chip” type device. Example absorbance data on a mesofluidic prepackaged module. Accordingly, a measurement device or user can correlate the measured change in absorbance with the amount of potassium in the aqueous sample.

The various embodiments described herein thus represent a technical improvement to conventional potassium measurement techniques. Using the techniques as described herein, an embodiment may use an indicator to measure potassium in solution using a method safer for users and more environmentally safe. This is in contrast to the use of formaldehyde with limitations mentioned above. Such techniques provide a faster and more accurate method for measuring potassium in an aqueous or liquid solution, while using less dangerous or harmful chemicals or reagents in potassium measurement.

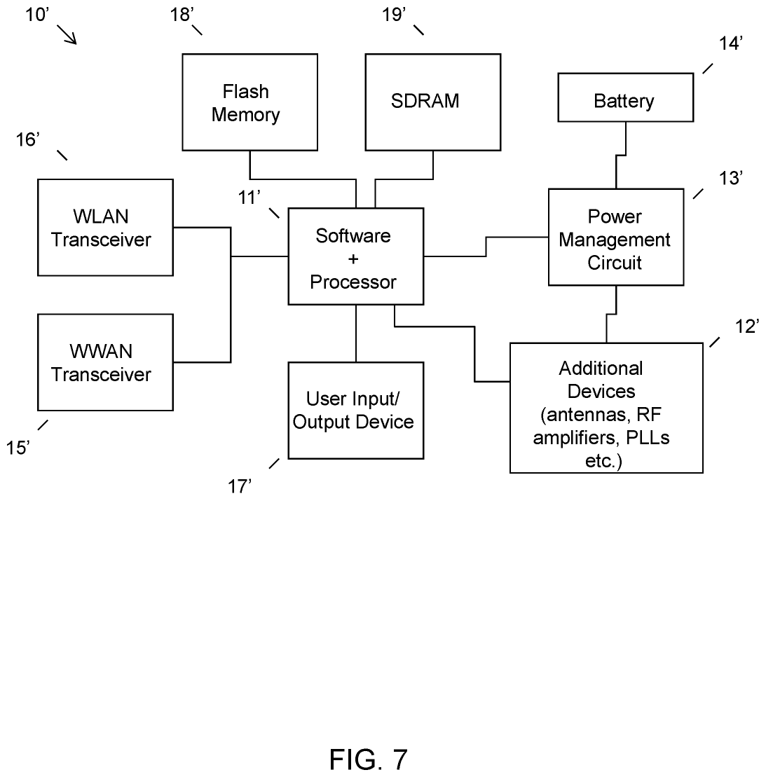

While various other circuits, circuitry or components may be utilized in information handling devices, regarding an instrument for measurement of potassium according to any one of the various embodiments described herein, an example is illustrated in FIG. 7. Device circuitry 10′ may include a measurement system on a chip design found, for example, a particular computing platform (e.g., mobile computing, desktop computing, etc.) Software and processor(s) are combined in a single chip 11′. Processors comprise internal arithmetic units, registers, cache memory, busses, I/O ports, etc., as is well known in the art. Internal busses and the like depend on different vendors, but essentially all the peripheral devices (12′) may attach to a single chip 11′. The circuitry 10′ combines the processor, memory control, and I/O controller hub all into a single chip 11′. Also, systems 10′ of this type do not typically use SATA or PCI or LPC. Common interfaces, for example, include SDIO and I2C.

There are power management chip(s) 13′, e.g., a battery management unit, BMU, which manage power as supplied, for example, via a rechargeable battery 14′, which may be recharged by a connection to a power source (not shown). In at least one design, a single chip, such as 11′, is used to supply BIOS like functionality and DRAM memory.

System 10′ typically includes one or more of a WWAN transceiver 15′ and a WLAN transceiver 16′ for connecting to various networks, such as telecommunications networks and wireless Internet devices, e.g., access points. Additionally, devices 12′ are commonly included, e.g., a transmit and receive antenna, oscillators, PLLs, etc. System 10′ includes input/output devices 17′ for data input and display/rendering (e.g., a computing location located away from the single beam system that is easily accessible by a user). System 10′ also typically includes various memory devices, for example flash memory 18′ and SDRAM 19′.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data. The disclosed system may be used in an embodiment to perform measurement of potassium of an aqueous sample or a sample.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for measuring potassium in a sample, comprising:
chelating an interferant, using an amount of sodium gluconate, in the sample;
mitigating an ammonium interference, using an amount of alkali hydroxide, in the sample; and
measuring, using a tetraphenylborate indicator, the amount of potassium in the sample by measuring a change in intensity of the absorbance of the chelated sample.

2. The method of claim 1, wherein the interferant comprises magnesium.

3. The method of claim 1, wherein the interferant comprises calcium.

4. The method of claim 1, wherein the alkali hydroxide is lithium hydroxide.

5. The method of claim 1, wherein the alkali hydroxide is sodium hydroxide.

6. The method of claim 1, wherein a concentration of the sodium gluconate is adjusted to the amount of the interferant in the sample.

7. The method of claim 1, wherein the change in intensity of the absorbance is proportional to a concentration of the amount of potassium in the sample.

8. The method of claim 1, wherein the change in intensity of the absorbance is measured using spectrophotometric methods.

9. The method of claim 1, wherein the measuring further comprises a gravimetric method.

10. The method of claim 1, wherein the sodium gluconate is introduced to the sample in a form selected from the group consisting of: a solution, a powder, and a prepackaged module.

11. A measurement device for measuring potassium in a sample, comprising:
a prepackaged module, wherein the prepackaged module comprises an amount of sodium gluconate;
a processor; and
a memory storing instructions executable by the processor to:
chelate an interferant, using the amount of sodium gluconate, in the sample;
mitigate an ammonium interference, using an amount of alkali hydroxide, in the sample; and
measure, using a tetraphenylborate indicator, the amount of potassium in the sample, using a photometric device, by measuring a change in intensity of the absorbance of the chelated sample.

12. The device of claim 11, wherein the interferant comprises magnesium.

13. The device of claim 11, wherein the interferant comprises calcium.

14. The device of claim 11, wherein the alkali hydroxide is lithium hydroxide.

15. The device of claim 11, wherein the alkali hydroxide is sodium hydroxide.

16. The device of claim 11, wherein a concentration of the sodium gluconate is adjusted to the amount of the interferant in the sample.

17. The device of claim 11, wherein the change in intensity of the absorbance is proportional to a concentration of the amount of potassium in the sample.

18. The device of claim 11, wherein the change in intensity of the absorbance is measured using spectrophotometric methods.

19. The device of claim 11, wherein the measuring further comprises a gravimetric method.

20. A product for measuring potassium in a sample, comprising:
a prepackaged module, wherein the prepackaged module comprises an amount of sodium gluconate;
a storage device that stores code, the code being executable by a processor and comprising:
code that chelates an interferant, using the amount of sodium gluconate, in the sample;
code that mitigates an ammonium interference, using an amount of alkali hydroxide, in the sample; and
code that measures, using a tetraphenylborate indicator, the amount of potassium in the sample, using a photometric device, by measuring a change in intensity of the absorbance of the chelated sample.
